(19) United States
(12) Patent Application Publication
(10) Pub. No.: US 2012/0172827 A1
(43) Pub. Date: Jul. 5, 2012

(54) REUSABLE, ADJUSTABLE-SIZE, POCKET DIAPER HAVING REPLACEABLE ELASTIC

(76) Inventor: Tereson R. Dupuy, Lafayette, LA (US)

(21) Appl. No.: 13/395,855

(22) PCT Filed: Sep. 13, 2010
(86) PCT No.: PCT/US10/48681

§ 371 Date: Mar. 13, 2012

Related U.S. Application Data
(60) Provisional application No. 61/241,952, filed on Sep. 13, 2009.

(51) Int. Cl. A61F 13/511

(52) U.S. Cl. 604/378

(57) ABSTRACT

A washably reusable diaper having water wicking inner layer, a water impervious outer layer, a space in between for insertion and removal of water absorbing pad and having an elongated pocket adjacent to the waist line of the diaper a nd an elongated pocket adjacent each leg line of the diaper. Disposed proximate each end of each elongated pocket is a means for releaseably attaching an elastic strip. In each of said pockets is an elastic strip having a number of mating means disposed along the length of the strip. Accordingly the length of elastic strip in each pocket may be selectively varied as the wearer of the diaper grows, for use on different wearers and the elastic strip may be easily removed and renewed when necessary, extending useful service life of the diaper.

### BACKGROUND OF THE INVENTION

1. Field of Invention

The invention herein disclosed and claimed relates generally to the field of washably reusable diapers having an inner panel of moisture permeable fabric, an outer panel of moisture impermeable fabric and space therebetween (commonly called a “pocket”) for removable insertion of moisture absorbing pad, commonly referred to as “pocket diapers”. More specifically the invention disclosed and claimed herein relates to a pocket diaper which is highly adjustable in size and has elastic about the leg and/or waist openings which may be facilely replaced when necessary.

2. General Background

The diapers of U.S. Pat. No. 6,579,273, which have come to be called “pocket diapers”, are well known. In such diaper the size of the waist and leg openings are designed to accommodate wearers of somewhat different size by virtue of fastening arrangement and by using strips of elastic about at least a portion of the waist and leg openings of said diaper. However, in such diaper the amount of adjustment was limited, and as a consequence a baby could be expected to go through at least three sizes of such diapers, small, medium and large, from infancy through the time they were toilet trained.

Attempts have been made to design a pocket diaper which is more adjustable in the size. In U.S. Pat. No. 7,629,501 to Labit et al a diaper is equipped with set of three horizontally disposed rows of snap fasteners going down the front of the diaper. By choosing which row of fasteners to use the rise of the diaper could be adjusted. At least in theory by shortening the rise of the diaper the size of leg opening of the diaper was reduced to allow for the diaper to be used on babies having smaller legs. However the folds of fabric gathered at the front of the diaper when a diaper of this design is used on smaller babies presents a risk of leakage under certain circumstances, primarily when the baby is laid on its stomach for long periods of time.

In another attempt to provide a washably reusable pocket diaper which is of adjustable size is seen in New Zealand Patent Number 549,433. There, the length of the elastic strip of the leg is made adjustable by disposition of the elastic strip in an elongated pocket adjacent the leg lines of the diaper. In said diaper one end of the elastic strip is permanently attached to one end of the elongated pocket, and the other end of the elastic strip is releaseably attached to the other end of the elongated pocket by a snap fastener. By disposing a plurality of snap members along the length of the elastic strip the operative length of the elastic adjacent each leg line may be varied. In the diaper of this design the length of the elastic strip at the waist of the diaper is not adjustable; the use of snap members along the length of the elastic strip presents cost and comfort disadvantages and the elastic strip (and snap members thereon) are not easily replaced when deteriorated by passage of time, repeated stretching and laundering.

It should be noted that one of the primarily desired advantage of a reusable diaper which is highly variable in size is to be able to use such diaper from the time a baby is born until it is toilet trained, namely for a period of 2 to 3 years. If the elastic strips of the waist and/or leg do not retain sufficient elasticity for 2 to 3 years, and the elastic strips, or the waist or leg lines, cannot be easily replaced, then the primarily desired objectives of using a reusable diaper which is highly variable in size will not, in the long run, actually be achieved, and the economy of purchasing such diaper will not be realized.

The invention disclosed and claimed herein seeks to address itself to some of the issues discussed above.

### OBJECTS OF THE INVENTION

The general object of the invention herein disclosed and claimed is to provide a washably reusable pocket diaper of improved design. Another object of the invention is to provide a pocket diaper which is highly adjustable in size, namely one where the size of the waist and leg opening may be independently varied across a wide range of sizes, generally being suitable for use on normal size infants through the time they are toilet trained. Another object of the invention herein disclosed and claimed is to provide elastic strips along the waistline and leg line which have an operative length that may be easily varied and said elastic strips may be easily replaced when necessary. Yet another object of the invention is to provide means to adjust the length of elastic used along the leg line which does not use a hard fastening members along the leg line of the diaper.

### SUMMARY OF INVENTION

The diaper of the invention has an inner panel of moisture permeable fabric, an outer panel of moisture impermeable fabric and a moisture absorbing pad disposed between said inner and outer panels. Said panels are of generally “hour-glass” shape having a wide front edge, a wide back edge and two side edges having a narrowed portion between generally intermediate said (wider) front and back edges. The inner panel and outer panels are attached at least about their side edges, may be attached at their front edges and rear edges except as may be necessary to create an opening for insertion and removal of moisture absorbing pad. The areas about the rear edge and the front edge which overlap when the diaper is wrapped around the waist of the wearer are fitted with mating fastening means (for fastening the diaper about the waist of the wearer). At the back edge of the diaper, the front edge of the diaper or both is an elongated pocket having two ends. Proximate each of said ends is a means for releaseably attaching an elastic strip. Slidably disposed in said elongated pocket is an elastic strip having mating means disposed along the length of said strip. In the preferred embodiment of the invention buttons may be used at each end of the elongated pocket and button hole used as mating means (along the elastic strip). The operative length of said elastic strip (on the waist of the wearer) may be varied by selection of button-holes (disposed along the length of the elastic strip) which are engaged with each of said buttons. The elastic strip may be marked with markings representing length between button-holes so as to facilitate selection of desired length of elastic strip between said buttons. Excess length of the elastic strip may be tucked between the inner and the outer panel of the diaper. The elastic strip may be easily replaced when worn or damaged by disconnecting both ends of the elastic strip from the diaper and inserting new elastic strip into the elongated pocket.

As to the leg portion of the diaper the side edges are incurved to form a narrowing crotch section of the diaper. The incurvate portions of the side edges from two (left and right) leg lines. Each leg line has an adjacent elongated pocket containing an elastic. Similar to described above, proximate each end of said elongate pocket is means for releaseably attaching the elastic strip disposed in that pocket at a plurality of mating fastening points along the length of the elastic strip. As with the pocket/elastic-strip configuration of the waist line the length of said elastic strip in the elongated pocket of each leg line may be varied by selection of fastening points (along the elastic strip), the fastening points may be numbered or otherwise marked to facilitate establishing a desired length, the elastic strip of each leg line may be easily replaced when the elastic is damaged, worn or otherwise needs replacement and excess elastic may be tucked between the inner and the outer panel of the diaper or cut off (as desired).

### BRIEF DESCRIPTION OF THE DRAWINGS

For further understanding of the nature and objects of the present invention, reference should be made to the following description of the preferred embodiment of the invention taken in conjunction with the accompanying drawings wherein:

FIG. 1 is a view of the inner panel of the preferred embodiment of the diaper of the invention.

FIG. 2 is a view of the outer panel of the preferred embodiment of the diaper of the invention.

FIG. 3 is a view of the inner panel of the preferred embodiment of the diaper of the invention with a portion of the inner panel folded back to expose elements of the invention underneath.

FIG. 4 is a cross-sectional view taken along sight lines 4-4 of FIG. 1.

### DESCRIPTION OF THE PREFERRED EMBODIMENT OF THE INVENTION

While the present invention will be described with reference to preferred embodiments, it will be understood by those who are skilled in the art that various changes may be made and equivalents may be substituted for elements thereof without departing from the scope of the invention. In addition, many modifications may be made to adapt a particular situation or material to the teachings of the invention without departing from the essential scope thereof. It is therefore intended that the present invention not be limited to the particular embodiments disclosed as the best mode contemplated for carrying out this invention, but that the invention will include all embodiments and legal equivalents thereof.

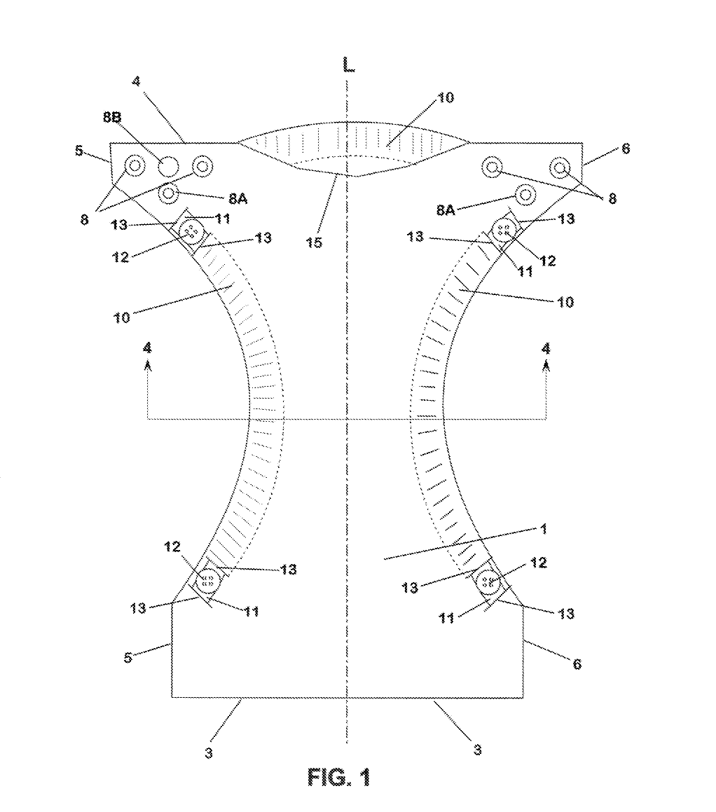

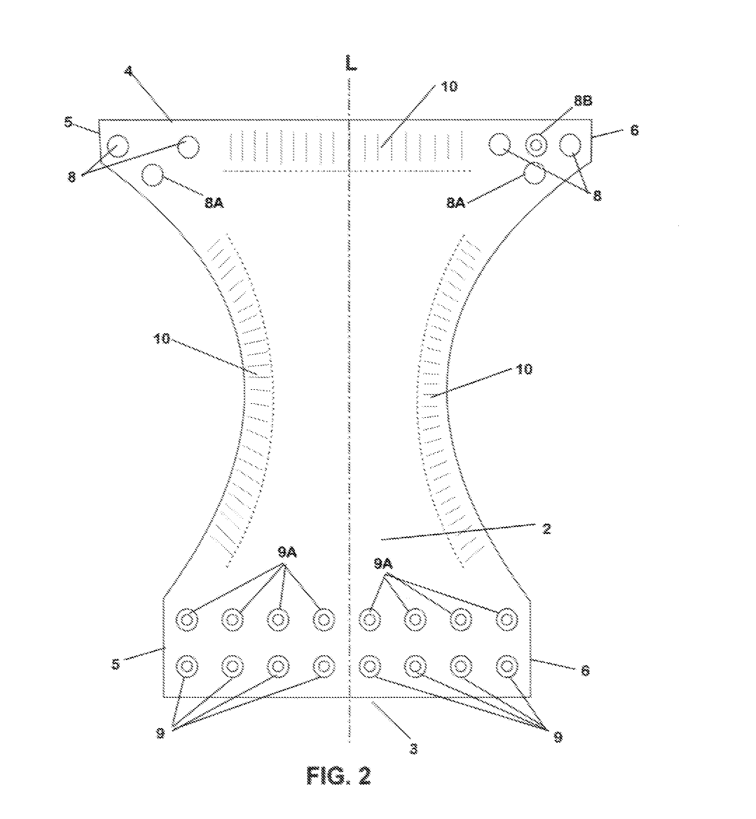

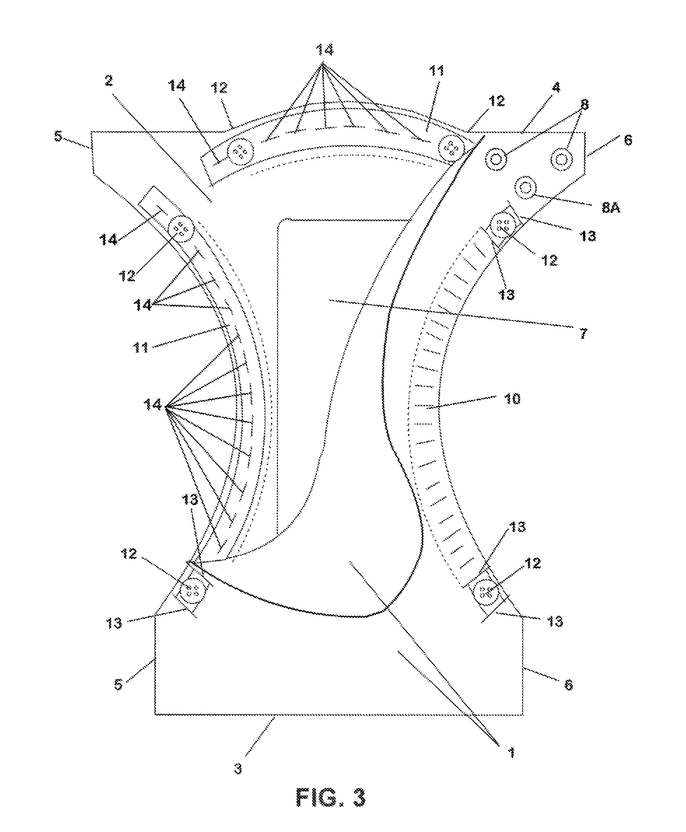

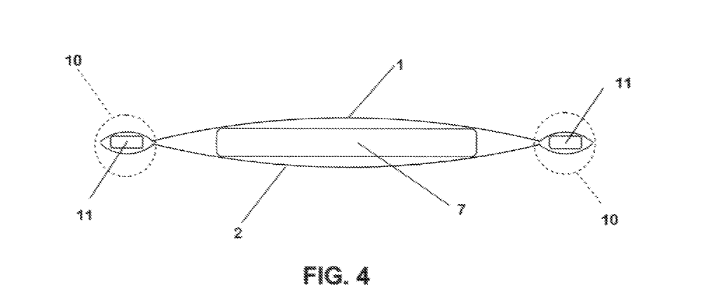

Referring to FIGS. 1, 2, 3 and 4 it is seen that the diaper of the invention herein disclosed and claimed has an inner panel 1, an outer panel 2 and pad 7 disposed between inner panel 1 and outer panel 2. As seen in FIGS. 1, 2 and 3 inner panel 1 and outer panel 2 are of a mating, generally “hour-glass” configuration having front edge 3, back edge 4 and side edges 5 and 6. Side edges 5 and 6 have an incurvate, narrowed crotch portion which is generally intermediate front 3 and back 4. Inner panel 1 and outer panel 2 are attached at least at side edges 5 and 6. Inner panel 1 and outer panel 2 may also be attached at front edges 3 and back edges 4 except as may be necessary to create an opening 15 for insertion and removal of pad 7. It will be appreciated by those skilled in the art that the inner and outer panels of the diaper may be attached about the entire periphery and an opening, for insertion and removal of pad 7, might constitute a slit of inner panel 1 or outer panel which is not at or adjacent to front edge 3 or back edge 4.

In the preferred embodiment of the invention inner panel 1 will be composed of a washably-reusable, light, soft, flexible fabric which is permeable to moisture, tends to wick moisture through it but itself retains little moisture, and feels dry to the touch even when somewhat wet. Fabric of knitted polyester pile, sometimes called “Polar Fleece” or simply “fleece”, is disclosed in U.S. Pat. No. 6,579,273 to have these characteristics in diapers and is preferred. Other fabric which have similar characteristics may be used in lieu of knitted polyester pile fabric. In the preferred embodiment of the invention outer panel 2 will be composed of a washably-reusable, light, soft, flexible fabric which is impermeable to moisture but may be, and preferably is, permeable to air. In the preferred embodiment of the invention pad 7 will be composed of a washable reusable or disposable material which is light, soft and flexible and tends to absorb and retain moisture. Materials including cotton and/or bamboo fibers have been found suitable for use for pad 7.

In the preferred embodiment of the invention the size of the waist opening of the diaper is made highly adjustable by two attributes, namely the means by which the diaper is overlappingly fastened about the waist of the wearer and the inclusion of at least one elastic strip slidably disposed in an elongated pocket adjacent the waistline of the diaper. The length disposed between fastening means at each end of the elongated pocket may be adjusted and the elastic replaced as desired.

As to the means by which the diaper is fastened about the waist of the wearer in the preferred embodiment of the invention, the front of the diaper (adjacent to front edge 3) and back of the diaper (adjacent to back edge 4) are fitted with means, such as mating male and female snap components 8 and 9 to overlappingly fasteners on a portion of said back to the front of the diaper (when lateral portions of said back are brought around the wearer to the front of the diaper), enabling the diaper to be secured about the waist of the wearer. In the preferred embodiment of the invention the front of the diaper will be fitted with a plurality of spaced apart fastener components 9 arranged in a row running transverse to the longitude L of the diaper (and parallel to front edge 3) so as to allow the diaper to be secured about wearers of different waist sizes. Similarly, in the preferred embodiment of the invention, the back of the diaper will have a plurality of fastener components 8 arranged in a row running traverse to the longitude L of the diaper (and parallel to back edge 4). In the preferred embodiment of the invention at least some of the fastening components at the back of the diaper will be reversed, number 8B of FIGS. 1, 2 and 3, so as to allow at least one lateral side of the back to be overlappingly fastened to the other lateral side of the back when both are brought about to the front of the diaper, thereby allowing the diaper to be secured over wearers having smaller waist size than the diaper could be secured on by overlappingly engaging portions of the back only with the front of the diaper. In the preferred embodiment of the invention both front and back of the diaper may be equipped with at least a second row of fastening components 8A and 9A so as to provide more secure closure of the diaper about the waist and some separate adjustment of size of the leg opening of the diaper.

As to the means by which a length elastic in the waist line of the diaper may be adjusted, it is seen in the FIGURES that in the preferred embodiment of the invention an elastic strip 11, is slidably disposed in elongated pocket 10 formed in at least a portion of the waist line of the diaper. In the preferred embodiment of the invention elongated pocket 10 is formed along part of back edge 4 by folding part of the back of outer panel 2 over itself and stitching same back to itself at a distance which is spaced somewhat apart from back 4. Those skilled in the art will recognize that an elongated pocket 10 could be formed along back edge 4, along front edge 3 or along both back edge 4 and front edge 3. Those skilled in the art will also recognize that elongated pocket 10 in inner panel 1, in outer panel 2 or between inner panel 1 and outer panel 2 in many other ways and all of such ways are intended to be comprehended by the scope of the invention claimed.

In elongated pocket 10 of the waist line of the diaper of the invention an elastic strip 11 is slidably disposed. The operative length of the elastic strip 11 in elongated pocket 10 may be adjustably selected by virtue of the fact that fastening means (for example buttons 12), attached to diaper itself, are provided proximate each end of pocket 10 and mating fastening means (for example button holes 14) are provided at various fastening points disposed along the length of elastic strip 11. In preferred embodiment the length of the button holes 14 will be disposed parallel to the length of the elastic strip 11. Length of elastic strip 11 between buttons 12 can be varied by selecting different button holes 14 to engage with buttons 12. By shortening the length of the elastic strip 11 between buttons 12, the waist of the diaper along elongated pocket 10 is gathered more closely and the diaper becomes suitable for use on wearers having smaller waists. Conversely by lengthening the length of elastic strip 11 between buttons 12, the waist of the diaper along elongated pocket 10 is gathered more loosely and the diaper becomes suitable for use on wearers having larger waists. In the preferred embodiment of the invention the elastic strip 11 will have markings, such as letters or numbers, to facilitate selection of desired length of elastic strip 11 between buttons 12. Advantageously it is also seen that by releaseably attaching each end of elastic strip 11 to buttons 12, the elastic strip 11 is easy to remove and replace when necessary. Those skilled in the art will recognize that use of buttons 12 and button holes 14 is only one of many means that elastic strip 11 might be attached to fastening means proximate each end of elongated pocket 10, mating halves of snap fasteners, various hook and eyes fastening means, including Velcro® and Aplix® might be used. While not preferred because of cost, and because disposition of hard fastening means on the elastic creates at least a risk of discomfort to the wearer, all such releaseable fastening means are intended to be embraced within the scope of the invention herein disclosed and claimed.

Similarly to the above described provision of an elastic strip at the waist line of the diaper of the invention, the diaper of the invention also includes (with or without elastic strip at the waist line of the diaper) an elongated pocket 10 containing slidably disposed elastic strip 11 disposed adjacent to each leg line (formed by the incurvate portions of side edges 5 and 6 of the diaper) of the diaper. At each leg line elastic strip 11, is slidably disposed in elongated pocket 10 and has an operative length which may be selected by selecting the length of elastic strip 11 which is disposed between mating fastening members, such as buttons 12, disposed proximate each of end of elongated pocket 10. Fastening members, such as buttons 12, are preferably attached to outer panel 2, but may be attached to inner panel 1 or may be attached to both panels. As described above each elastic strip 11 of each leg line will have at various fastening points, such as button holes 14, disposed along the length of said elastic strip 11. If button holes 14 are used they are preferably disposed with length parallel to length of elastic strip 11. As described above, by choosing different fastening points, such as button holes 14, of elastic strip 11, to engage with fastening members, such as buttons 12, the length of the elastic strip disposed between fastening members, such as buttons 12, may be selectably varied as desired. As described above other means of releaseably engaging fastening points along elastic strip 11 with fastening means proximate the ends of elongated pockets 10 will be known to those skilled in the art and are intended to be embraced within the scope of the invention disclosed and claimed herein.

In the preferred embodiment of the invention elongated pockets 10 along the leg lines are formed by attaching inner panel 1 to outer panel 2 at a distance which is spaced somewhat inboard (offset from) but will be generally parallel to incurvate portion of side edges 5 and 6. Those skilled in the art will recognize that the elongated pocket 10 might be formed by various other means all of which are intended to be embraced within the scope of the invention disclosed and claimed herein.

Proximate each end of elongated pocket 10 will be an opening, such as slit 13, in inner panel 1 so as to provide access to fastening members, such as buttons 12, and provide a means whereby excess length of elastic strip 11 (namely the length of elastic strip 11 which extends beyond buttons 12) may be tucked under inner panel 1 or cut off if desired. The length of elastic strips 11 disposed in the elongated pockets 10 of the leg lines of the diaper may be shortened to decrease the size of the leg openings of the diaper to accommodate wearers of smaller leg size or lengthened to increase the size of the leg opening of the diaper to accommodate wearers of larger leg size. To a degree, decreasing the size of the waist opening also operates, in combination with adjustable elastic strip 11 of the leg lines of the diaper, to decrease the size of the leg openings as does using different fastening points along the second row of fasteners 9A and 9B at the front and back of the diaper; all together forming a pocket diaper of highly adjustable size and having facilely replaceable elastic stripping. It should also be appreciated that by making the elastic of both the waist and legs of the diaper facilely removable and replaceable the diaper of the invention can, by replacement of inexpensive elastic strips, be expected to provide longer service life than it might otherwise have if the elastic stripping of the diaper were not replaceable.

While the above description contains certain specifics, these should not be construed as limitations on the scope of the invention, but rather as an exemplification of one preferred embodiment thereof. Accordingly, the scope of the present invention should be determined not by the embodiment(s) illustrated, but by such claims and their legal equivalents as may be granted upon the disclosure herein made.

What is claimed is:

1. A adjustable size diaper comprising:
a) an outer panel comprised of a water-impervious fabric, having front and rear body portions connected by incurvate crotch portion disposed between, wherein said front and rear body portions each have an upper edge, said upper edges together, in use, defining a waist-line and said incurvate crotch portion having a first side edge defining, in use, a first leg-line and a second side edge defining, in use, a second leg-line;
b) an inner panel comprised of a water-wicking fabric, having a contour substantially matching the contour of said outer panel;
c) a pocket-like space between said outer panel and said inner panel for insertion and removal of a water absorbing pad between said outer and inner panels;
d) adjacent each of said first and second leg lines an elongated fabric pocket having a first end and a second end;
e) attached to at least one of said outer or inner panels disposed proximate to each of said first and second end of each elongated pocket a fastening member for releasebly attaching an elastic strip having mating attachment means;
f) slidably disposed in each of said elongated pockets an elastic strip;
g) disposed along the length of each of said elastic strips a plurality of means for mating attachment a selected portion of each of said elastic strips to a selected fastening members; and,
h) an opening proximate each said fastening members for access to each of said fastening members and said elastic strips.

2. The diaper of claim 1 further comprising:
f) means for selectively adjusting the length of said waist-line comprising an elongated pocket, fastening members proximate each end of said elongated pocket and disposed in said elongate pocket an elastic strip having a plurality of means disposed along for the length of said elastic strip for releaseably attaching a select portion of said elastic strip to a selected fastening member.

3. The diaper of claim 1 or 2 wherein said at least some of said fastening members comprise a button and said means for mating attachment of said elastic strip to said fastening members comprise a button hole.

4. The diaper of claim 1 or 2 wherein said at least some of said fastening members comprise a hook means and said means for mating attachment of said elastic strip to said fastening members comprise a loop means.

5. The diaper of claim 1 or 2 wherein said at least some of said fastening members comprise a one part of a snap fastener and the means for mating attachment of said elastic strip to said fastening members comprises the mating part of said snap fastener.
